(19) United States
(12) Patent Application Publication
(10) Pub. No.: US 2012/0258527 A1
(43) Pub. Date: Oct. 11, 2012

(54) BIOREACTOR

(76) Inventor: Ian Malcolm Wright, Stapylton (AU)

(21) Appl. No.: 13/381,510

(22) PCT Filed: Mar. 9, 2010
(86) PCT No.: PCT/AU2010/000261

§ 371 Date: Jun. 29, 2012

(30) Foreign Application Priority Data
Mar. 9, 2009 (AU) 2009901008

(51) Int. Cl. C12M 1/42

(52) U.S. Cl. 435/292.1

(57) ABSTRACT

A bioreactor including a substantially horizontal containing vessel having a base and at least one upstanding side-wall, at least one inlet located at a lower portion of the containing vessel, at least one outlet located at an upper portion of the containing vessel, an upper wall having a reflective material associated there with, at least one light source associated with the upper wall, and at least one light tube associated with each light source extending substantially the length of at least one sidewall of the vessel, the light tube provided adjacent an upper edge of the at least one sidewall and having a reflective lining to direct light from the light source across the upper wall.

### FIELD OF THE INVENTION

The present invention relates to bioreactors and in particular bioreactors for batch growth of biomass for various uses.

### BACKGROUND ART

For many years, bioreactors have been considered to be an ideal technical solution for the growing of feedstock for the extraction of oils. Bioreactors have also been suggested as a method to remove carbon dioxide from industrial emissions. The practical use of bioreactors has had limited success due to the cost of building and maintaining the bioreactor vessels and also the difficulty in removing the products from the reactor vessel once mature.

Bioreactors degrade contaminants in water with microorganisms through attached or suspended biological systems. In suspended growth systems, such as activated sludge, fluidized beds, or sequencing batch reactors, contaminated ground water is circulated in an aeration basin where a microbial population aerobically degrades organic matter and produces CO₂, H₂O, and new cells. The cells form a sludge, which is settled out in a clarifier, and is either recycled to the aeration basin or disposed. In attached growth systems, such as upflow fixed film bioreactors, rotating biological contactors (RBCs), and trickling filters, microorganisms are established on an inert support matrix to aerobically degrade water contaminants.

The present invention has been specifically devised in order to provide a low-cost bioreactor with simple, effective design capable of providing a continuous or reusable process.

It will be clearly understood that, if a prior art publication is referred to herein, this reference does not constitute an admission that the publication forms part of the common general knowledge in the art in Australia or in any other country.

### SUMMARY OF THE INVENTION

The present invention is directed to a bioreactor, which may at least partially overcome at least one of the abovementioned disadvantages or provide the consumer with a useful or commercial choice.

With the foregoing in view, the present invention in one form, resides broadly in a bioreactor including
a. a substantially horizontal containing vessel having a base and at least one upstanding sidewall;
b. at least one inlet located at a lower portion of the containing vessel;
c. at least one outlet located at an upper portion of the containing vessel;
d. an upper wall having a reflective material associated there with;
e. at least one light source associated with the upper wall; and
f. at least one light tube associated with each light source extending substantially the length of at least one sidewall of the vessel, the light tube provided adjacent an upper edge of the at least one sidewall and having a reflective lining to direct light from the light source across the upper wall.

The present invention therefore is intended to provided a device for culturing cellular biological material in the form of solid particles in which the said solid particles are brought into contact with a liquid culture medium under optimum growth conditions and additionally ensure a simpler biomass extraction procedure.

The bioreactor of the present invention can be adapted for any use, but is particularly well-suited for growing carbon dioxide sequestrating biological material, methane digestion, fuel production or water production. Any biological material or growth culture medium may be used.

The bioreactor of the present invention will normally include a liquid culture medium. The biological material will normally be referred to as “solid particles” only in order to distinguish them from the liquid culture medium.

The bioreactor containing vessel of the present invention will typically be substantially rectangular. The container vessel is preferably configured as a tank or similar of a standard height and dimension. In this form, multiple vessels may be stacked one above the other to form towers of multiple vessels to minimise the footprint area of the bioreactors. In this way, a large capacity bioreactor farm can be housed in a smaller area.

Alternatively the bioreactor containing vessel may be configured substantially as a table or similar containing vessel position at a height above ground level, normally on one or more legs.

The containing vessel may be manufactured of any substance. However, it is preferred that the containing vessel is manufactured of material which is opaque in order to prevent light entering the containing vessel other than that from the light source.

The containing vessel will typically be watertight in order to contain the liquid culture medium and biological material.

According to its most preferred form, the containing vessel will normally have a substantially planar base wall and four upstanding sidewalls configured in two opposed pairs. There may be arcuate portions provided at the joining points between the base wall and each of the upstanding sidewalls in order to minimise “dead” areas in the bioreactor.

It is further preferred that the containing vessel of the present invention is provided with a diffuser or similar device through which a gas or second liquid may enter the containing vessel. Preferably, the diffuser will be configured as a porous mat, however other configurations such as a plurality of pipes or tubes with openings therein, may be used.

The diffuser will preferably be linked to a supply of gas or second liquid which may be beneficial to the growth of the biological material. An example of one such gas which may be beneficial to the growth of the material is carbon dioxide. Any gas or secondary liquid of a beneficial nature will typically be metered into the containing vessel in an amount which is sufficient to enhance or positively affect the growth of the biological material, but to reduce waste or oversupply.

In its most preferred form, the diffuser will typically be arranged to substantially cover the base wall of the containing vessel in order that all portions of the bioreactor are affected by any gas or second liquid. As a consequence of the input of gas or secondary liquid, the liquid culture medium already present in the containing vessel may be agitated or stirred.

The bioreactor of the present invention also has an inlet located in a lower portion of the containing vessel. The inlet will typically be located in or through a sidewall preferably on one side of the containing vessel. The inlet may be directed toward or face toward an opposite wall or portion of the wall. Preferably, the inlet will be located substantially opposite the outlet in order to help flush liquid and/or biomass toward the outlet.

The inlet will normally be provided with a valve to prevent flow into and out of the containing vessel and to regulate the flow into the containing vessel.

The bioreactor of the present invention also has an outlet located in an upper portion of the containing vessel. As mentioned above, the outlet will typically be substantially opposite the inlet. In the preferred rectangular containing vessel configuration, the outlet is located at approximately midway along one of the sidewalls of the containing vessel and the inlet is preferably located in a corner of the opposite wall and directed substantially parallel with that wall in order to create a vortex effect when the inlet is open and liquid is entering the containing vessel through the inlet.

The outlet is located in or through an upper portion of a sidewall. The outlet will typically be associated with a pump in order to assist outflow of material from the containing vessel. A vacuum pump, or similar, in order to draw the outflow out of the containing vessel through the outlet, particularly the mature biological material, is particularly preferred.

The containing vessel may be provided with a secondary outlet located in or through the base wall in order to drain the containing vessel. Typically, this secondary outlet will be used in order to remove a portion of the liquid added during the extraction phase in order to lower the level of liquid to allow for fresh growth of biological material in the growth phase.

The bioreactor of the present invention is provided with an upper wall, typically in the form of a lid or similar. It is preferred that the containment area of the bioreactor is substantially closed in order to optimise growth of the biological material. However, the interior of the containing vessel should be accessible for maintenance for example. Therefore, the upper wall or lid will typically be removable from the containing vessel.

The upper wall or lid will typically overlie the sidewalls of the containing vessel. Normally, the upper wall or lid will have a shape which matches the shape of the containing vessel or at least, match the shape of the upper terminus of the sidewalls of the containing vessel.

The upper wall or lid will usually have a depending wall to receive the upper terminal edges of the sidewall of the vessel in order to positively locate the upper wall or lid relative to the containing vessel. The depending wall will also assist with maintaining the upper wall or lid on the containing vessel against dislodgement as the upper wall or lid will then have to be lifted vertically before lateral movement is allowed.

In the alternative, other location and securing means may be provided such as number of downward finger extensions or protrusions which will similarly engage with the sidewalls of the containing vessel to minimise or prevent dislodgement of the upper wall or lid.

Preferably, the upper wall or lid will be manufactured of a transparent or translucent material such as a plastic material. Acrylic plastic is particularly preferred due to its high transmissivity of electromagnetic radiation and particularly light.

Light scattering agents will preferably be incorporated into the upper wall or lid and normally, embedded in the material used to manufacture the upper wall or lid. The light scattering agents may be mechanical/physical such as reflective flecks or optical devices such as portions of diffusive material or diffusive areas. It is preferred that the upper wall or lid be a substantially planar member. It is also preferred that the upper wall or lid be of sufficient thickness in order to be self-supporting across the dimension of the containing vessel. This thickness will of course be adjusted according to the material of construction.

It is particularly preferred that the upper surface and at least some of the edges of the upper wall or lid are provided with, normally coated with, a reflective material in order to contain and direct light from the light source into the containing vessel.

At least one light tube may be provided at one side of the preferred rectangular upper wall or lid and preferably the other three edges of the upper wall or lid will be provided with the reflective material as well as the entire upper surface of the upper wall or lid. The edge of the upper wall or lid with the light tube will normally not be provided with reflective material.

There may be one or more light tubes associated with a containing vessel. Normally, a light tube will be located on each of a pair of opposed sides of the vessel. However, the number of light tubes provided will normally depend upon the size of the containing vessel, its depth or other parameter. A sufficient number of lighting tubes will normally be provied to minimise “dead areas” within the containing vessel.

Alternatively, the upper wall may be shaped to direct any light downward. For example, the upper wall or lead may be partially arcuate across the containing vessel or partially spherical.

In both alternatives, the light tube is located at a level which is above and immediately adjacent the upper terminal edge of the sidewall of the containing vessel and in association with the upper wall such that the light can be transmitted across the upper wall or lid within the bounds of the reflective material. In the first alternative, the light tube will normally be provided at a level which is coplanar with the upper wall or lid. The bioreactor of the present invention includes a light source and any light source may be used.

According to a particularly preferred form, the light source will typically include a plurality of coloured light emitting diodes mounted on a support, the light emitting diodes chosen to provide predetermined spectral wavelengths and an optical device to transmit the emitted light.

According to an alternative embodiment, the lighting system includes a plurality of light emitting diodes mounted on a support, the light emitting diodes chosen to provide optimum spectral wavelengths to the biological material and an optical device to transmit the emitted light for illumination of the biological material. The lighting apparatus of the present invention includes a support which according to the most preferred embodiment used on the cylindrical light tube, is typically circular. Normally, the support of the present invention will be a part of or form a bottom or top plate for the bioreactor.

The lighting apparatus of the present invention is normally a powered electrical apparatus and as such, requires electrical connections to a power source. The electrical connections normally extend at least into the support from below (or above) as separation from the biological material which is to be cultured in the bioreactor is preferred as the biological material is normally at least partially liquid.

The preferred method of providing electrical power to the light emitting diodes is through the provision of power supply support “legs”. Typically, each leg will be a positive or negative terminal connected in turn to the power source. Typically, the support legs will be extend perpendicularly to the plane of the support and extend at least partially into the support.

According to a particularly preferred embodiment, an inner terminus of the support legs (defined relative to the support and being on the side of the support with the LED's) will preferably stand slightly proud of an upper surface of the support. The respective inner termini may be used as positive location means for a cover or the optical device.

Preferably, there will be a number of positive terminal legs and a lesser number of negative terminal legs. For the most preferred embodiment, for terminal legs will be provided and three of these will be positive terminal legs with only one being a negative terminal leg. All of the terminal legs will have appropriate connections to the light emitting diodes. The connection configuration is further discussed below.

The support of the present invention will typically be provided in one of two preferred implements namely, as a substantially planar plate or as a tiered system. When provided as a substantially planar plate, the support will normally form the bottom or top plate for the preferred cylindrical bioreactor.

When provided as a tiered system, there will typically be an outer plate and a spaced inner plate. Preferably, the outer plate is of larger dimension than the inner plate. Normally, the inner plate will be maintained above the outer plate by a shoulder wall which will preferably be continuous in order to define an internal space between an inner surface of the outer plate, the outer surface of the inner plate and the inner surface of the shoulder wall.

Typically, the electrical connection system between the terminal legs and the light emitting diodes will be located either in the space between the respective plates or alternatively, below the outer plate.

Normally, the light emitting diodes will be mounted on an appropriate mounting plate which is itself then subsequently mounted on the support. Preferably, the electrical connections with the light emitting diodes will extend through the support to connect with an outer side of the mounting plate.

According to a preferred embodiment, the mounting plate will be a printed circuit board containing electrical connections for the light emitting diodes.

Normally, the mounting plate will allow replacement of the light emitting diodes. The mounting plate will normally be substantially centrally mounted relative to the support and will maintain the light emitting diodes facing inwardly toward the bioreactor.

Normally, the inner plate is smaller diameter than the outer plate. The provision of the larger dimension outer plate and a shoulder wall provides a section of the support, namely the shoulder wall and the inner plate, which may be used to positively locate the support with in the bioreactor. For example, normally a terminal end of the outer expandable growth containment portion can be located about the shoulder wall and inner plate and abutting the outer plate. A circumferential clamp means may then be used to clamp or otherwise secure the outer expandable growth containment portion relative to the shoulder wall.

It is also anticipated that the lighting system of the present invention may be provided as a unit separately from the apparatus in relation to which it is used.

As a preferred embodiment is particularly adapted to use with a bioreactor, the lighting system may not be attached to the bioreactor, but rather to the light tube. In this regard, the light emitted by the light emitting diodes may be transmitted in any appropriate manner to light tube.

The apparatus and system of the invention includes a plurality of light emitting diodes (LEDs). Any number of LEDs may be provided but, a square number is preferred for ease of electrical connection.

As stated above, the number and combination of colours of the LEDs provided will be selected to provide an optimum wavelength of emitted light. Normally, the optimum wavelength will be within a particular range of wavelengths and the range will be chosen according to the biological material or plant material to be cultured.

For example, even though photosynthesis (and thus growth) is almost equally efficient across the entire visible spectrum from 400 to 700 nm, chlorophyll absorbs maximally in the blue and red portions of the spectrum.

As a further example, LED's emitting a lot of short wavelengths (380-470 nm) have been found to induce astaxanthin accumulation of up to 5 to 6% per dry cell, although the induction course the suppression of cell growth in the photosynthetic microalgae Haematococcus pluvialis, a potential source of astaxanthin. Therefore, the growth of this microalgae under the elimination of red LEDs (λmax=625 nm) to allow for cell growth without inducing a higher level of astaxanthin accumulation, and in switching to elimination with blue LEDs (λmax=470 nm) at a high light intensity to induce a high level of astaxanthin has been used effectively.

It is also a particularly preferred feature of the present invention that the cluster of LEDs are adjustable in order to adjust the available applied wavelengths of light during the various growth phases of the biological medium as required to stimulate or encourage growth during that particular growth phase.

Normally, the component LEDs used in the cluster for a particular application will be chosen in order to provide a range of wavelengths and normally, the individual LEDs will be actuable to allow adjustment of the wavelengths emitted from a particular cluster. For example, an LED cluster of the present invention may include LEDs of each of the component colours, for example, red (λmax=625 nm), green (λmax=525 nm), blue (λmax=470 nm), blue-purple (λmax=410 nm), purple (λmax=380 nm), ultraviolet ((λmax=less than 380 nm) and infrared (λmax=greater than 625 nm). By having particular LEDs in the cluster in the light and others not emit light, a wavelength range of any desired wavelength can be emitted from the apparatus. Not only that, but by switching the particular combinations which are lit and unlit, the wavelength range can be adjusted during the growth phases of the material.

As outlined above, the plurality of LEDs are normally mounted on a printed circuit board in an array, the array is normally a regular array and according to a particularly preferred embodiment, includes nine or 16 LEDs.

Typically, the LEDs are connected in the most efficient method electrically. Normally, the negative terminal will be connected to each of the rows of LEDs according to a pattern similar to the shape of the Greek letter psi (Ψ). Other configurations may be used.

The apparatus of the present invention also preferably includes an optical device to transmit the emitted light. In its simplest form applied to use in the bioreactor field, the optical device may be a transparent or translucent cover placed over the LED cluster in order to emit the light directly into the bioreactor.

Other more advanced optical devices may be used for particular applications. For example, the optical device may be a lens which is adapted to focus or disperse the emitted light. The optical device may therefore be a convex or concave shape depending upon requirements.

Alternatively, the light emitted by the LED cluster may be conveyed, transmitted or spread using a fibre-optic cable. In order to be used in this manner, the emitted light may need to be focused to a smaller cross-section beam, and in this instance, a focusing lens may be used.

More than one lighting apparatus according to the present invention may be used, particularly in the bioreactor field where as the biological material grows, it will usually absorb the light and prevent the light from reaching the more central portions of the biological mass. In this instance, it may be beneficial to agitate the biological mass in the bioreactor in order to expose all areas to the emitted light or alternatively, provide enough lighting apparatus spaced around the bioreactor to expose all portions of the biological mass to the emitted light.

In a further alternative form, the emitted light may be dispersed. One mechanism for doing this is providing a shaped reflector at an opposite end of the light tube to the light source in order to disperse the light. A further example of a mechanism for dispersing the light is to score the outside of a fibre-optic cable used to transmit the light, in channels in order to allow the light to escape from the fibre-optic cable.

A fibre optic cable may be provided within the light tube or alternatively, it may be the light tube.

The bioreactor of the present invention is provided with at least one light tube. The light tube will typically be located in association with the upper wall or lid in order to direct light into or across the upper wall or lid and then the reflective portions of the upper wall or lid will direct the light downward into the containing vessel. For example, the light tube may be located extending along one edge of and abutting the upper wall or lid where the upper wall or lid is plain or alternatively if the upper wall or lid is partially arcuate, the light tube may be located approximately centrally across the arc, below the upper wall or lid.

The light tube will preferably be provided with reflective portions to allow the transmission of light along the length of the tube and also at least one section of the light tube will lack reflective material in order to allow the light to be directed outwardly through the at least one section. Normally, the at least one section will correspond to the edge of the upper wall or lid where it is planar with the rest of the light tube being provided with a reflective lining or coating, or there may be more than one section spaced radially around the light tube where the upper wall or lid is arcuate.

The light tube will typically be cylindrical but may have any shape. A portion of the light tube, normally the at least one section without reflective material will abut an edge of the upper wall or lid and thereby transmit light from the light tube into the thickness of the upper wall or lid.

The light source will normally be located at one end of an elongate, hollow and typically cylindrical light tube and the opposite end of the light tube will normally be capped with reflective material. The cap may be shaped and normally will have a convex shape in order to scatter or disperse incident light.

The light tube may be hollow or alternatively, the light tube may be a solid rod such as that used in fibre optics. By providing a solid rod with an external reflective layer, a light tube can be formed. Where the light tube is hollow, it will typically be internally reflective.

There will typically be one light tube for each light source. Preferably, there will be more than one light source and associated light tube provided for each upper wall or lid. Normally, one light source and tube assembly will be provided on each of a pair of opposed sides of the upper wall or lid to reduce “dead” or darkened sections in the containing vessel although a single light source and tube assembly may be provided for smaller bioreactors.

The bioreactor of the present invention is preferably operable in two modes, namely a growth mode and an extraction mode.

In the growth mode, the valve in the inlet to the containing vessel will normally be closed. Gas or other material beneficial to the growth of the biological material will normally be bubbled through the liquid growth media contained in the containing vessel via the diffuser. The liquid growth media may be seeded with the biological material. The level of liquid growth media and any biomass layer in the containing vessel will normally be below the level of the outlet. The outlet pump will normally be off In this configuration, the biological material will typically float on the liquid growth media as it grows. Once a satisfactory amount of the biological material has accumulated in the floating layer, the bioreactor may be switched to the extraction mode.

In the extraction mode, the gas supply to the diffuser will be stopped and the light source may also be switched off. The inlet will normally be opened and liquid, normally water will be pumped into the containing vessel to raise the level in the vessel. Normally, this will be done at a low flow rate in order to minimise disruption to the layer of biological material. As the biological material via reaches the level of the outlet, the outlet pump will typically be switched on in order to draw the biological material layer from the top of the liquid. Minimal liquid will preferably be removed in the extraction mode.

Once extraction of the biological material layer has been completed, a portion of the liquid in the containing vessel may be drained in order to lower the level below the outlet in order to facilitate growth of the biological layer once more. The bioreactor then typically enters the growth mode again. By cycling through the growth mode and extraction mode, typically with minimal or no operator interaction, the bioreactor may function through numerous batch growth and extraction modes relatively automatically.

### BRIEF DESCRIPTION OF THE DRAWINGS

Various embodiments of the invention will be described with reference to the following drawings, in which:

FIG. 1A is a view from above of a preferred form of upper wall used with a bioreactor of the present invention having a light source and light tube.

FIG. 1B is an end view of the upper wall illustrated in FIG. 1A from the direction A-A as illustrated in FIG. 1C.

FIG. 1C is a view from the side of the upper wall illustrated in FIG. 1A.

FIG. 2 is a sectional side view of a bioreactor according to a preferred embodiment of the present invention in the growth mode.

FIG. 3 is a sectional side view of a bioreactor according to a preferred embodiment of the present invention in the extraction mode.

FIG. 4 is an isometric view of a bioreactor according to a preferred embodiment of the present invention with upper wall removed and the layers in the bioreactor sectioned.

FIG. 5 is an isometric view of the bioreactor of a preferred embodiment of the present invention with a single light tube.

FIG. 6 is an isometric view of the bioreactor of a preferred embodiment of the present invention with a pair of light tubes.

FIG. 7A is a view from above of an upper wall used with a bioreactor of the present invention having a pair of light sources and a pair of light tubes.

FIG. 7B is an end view of the upper wall illustrated in FIG. 7A from the direction A-A as illustrated in FIG. 7C.

FIG. 7C is a view from the side of the upper wall illustrated in FIG. 7A.

FIG. 8 is a sectional side view of a bioreactor having a pair of light sources and a pair of light tubes in the extraction mode.

FIG. 9 is a sectional side view of a bioreactor having a pair of light sources and a pair of light tubes in the growth mode.

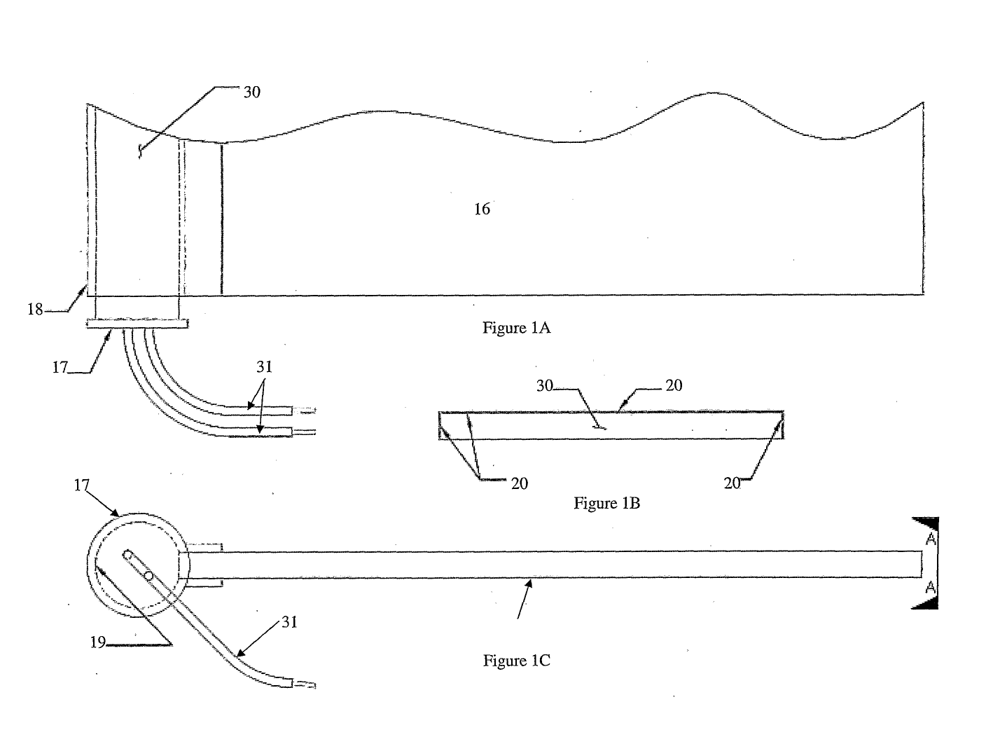

### DETAILED DESCRIPTION OF THE PREFERRED EMBODIMENT

According to a preferred embodiment, a bioreactor for culturing cellular biological material is provided.

The preferred form of bioreactor 10 illustrated in the Figures includes a rectangular, substantially horizontal containing vessel 11 having a base 12 and four upstanding sidewalls 13. The vessel 11 has an inlet 14 located at a lower portion and an outlet 15 located at an upper portion. An upper wall or lid 16 is provided having a reflective material 20 associated therewith. A light source 17 is associated with the upper wall 16, and a light tube 18 is associated with each light source 17, the light tube 18 extending substantially the length of one sidewall 13 of the vessel 11 coplanar with the upper wall 16 and having a reflective lining 19 to direct light from the light source 17 across the upper wall 16.

The bioreactor of the present invention includes a liquid culture medium 21 in the vessel 11. The biological material will normally be referred to as “solid particles” or “biomass” in order to distinguish them from the liquid culture medium only.

According to the illustrated embodiment, the container vessel 11 is configured substantially as a table or similar containing vessel position at a height above ground level, normally on legs 22. The containing vessel illustrated is approximately 2m by 3m by 150 mm in height.

The containing vessel 11 of the illustrated embodiment is provided with a diffuser or porous mat 23 through which a gas or second liquid may enter the containing vessel 11.

The porous mat 23 is linked to a supply of gas 24 via a connection conduit 25 which is beneficial to the growth of the biological material. An example of one such gas which may be beneficial to the growth of the material is carbon dioxide.

In its most preferred form as illustrated, the porous mat 23 is arranged to substantially cover the base wall 12 of the containing vessel 11 in order that all portions of the liquid culture medium 21 in the vessel 11 are affected by any gas.

The inlet 14 is located through a sidewall 13 on one side of the containing vessel 11. As illustrated, the inlet 14 is located substantially opposite the outlet 15 in order to help flush liquid or biomass toward the outlet 15 during the extraction phase.

The inlet 14 is provided with a valve 26 to prevent flow into and out of the containing vessel 11 and to regulate the flow into the containing vessel 11.

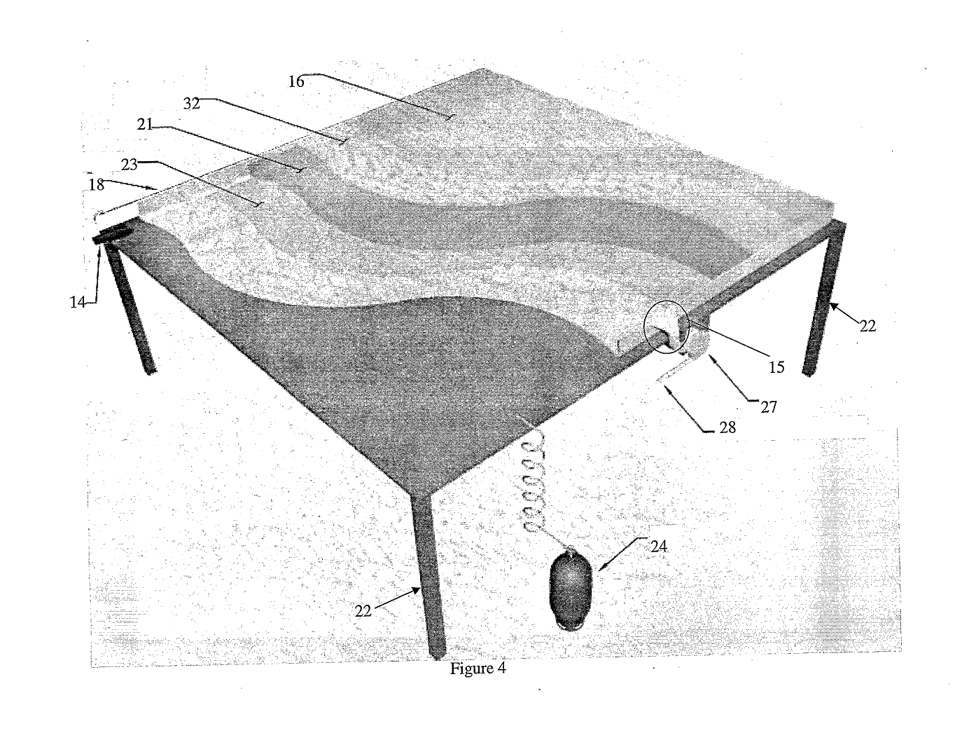

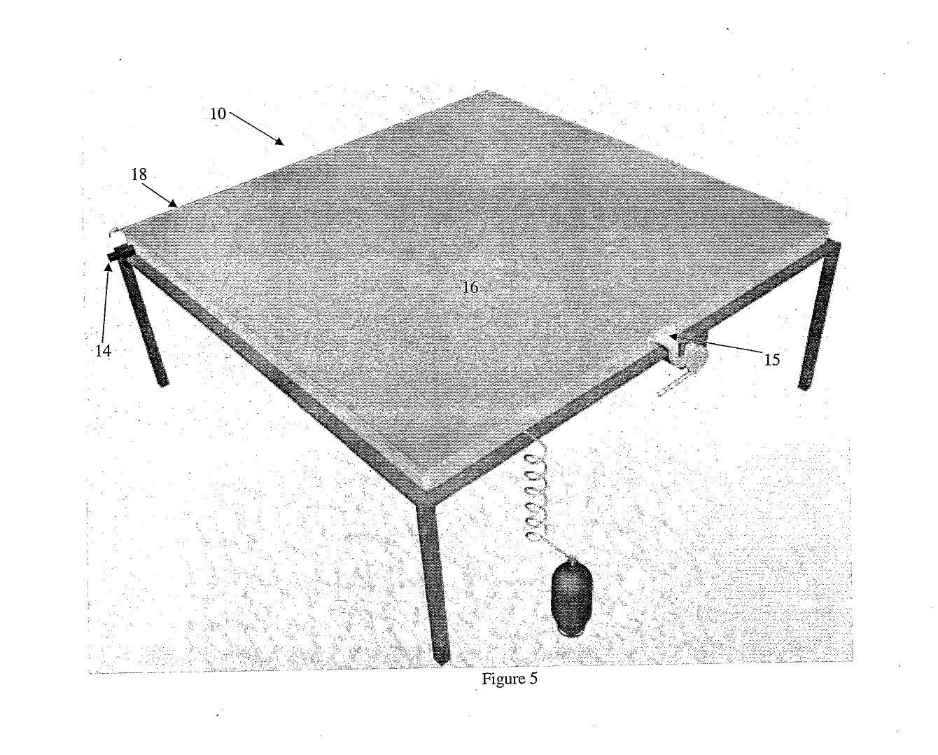

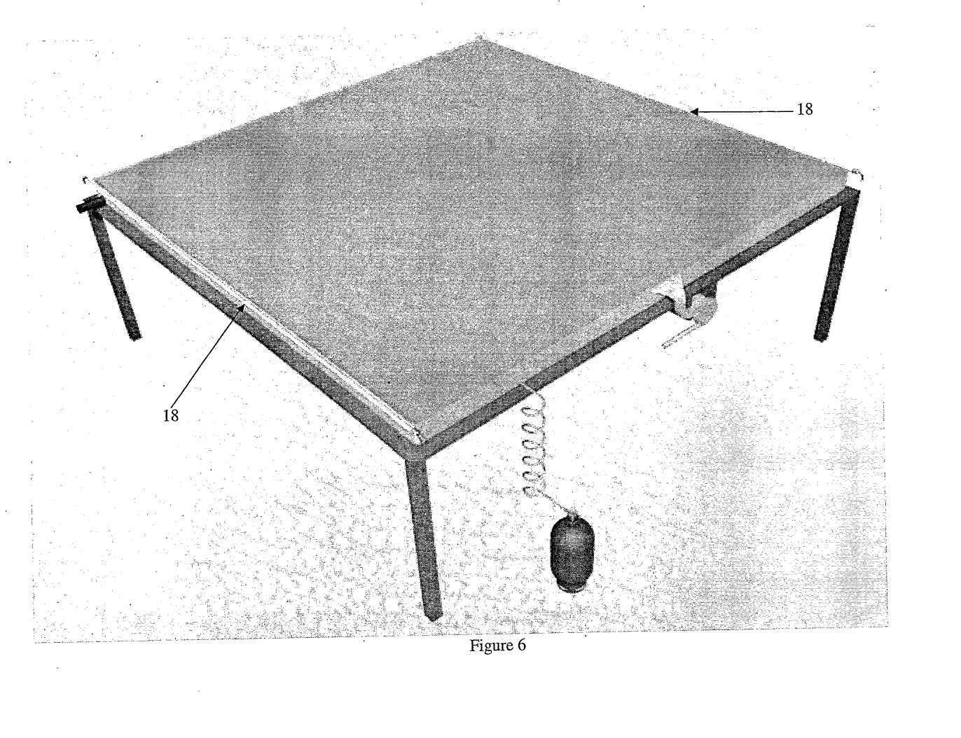

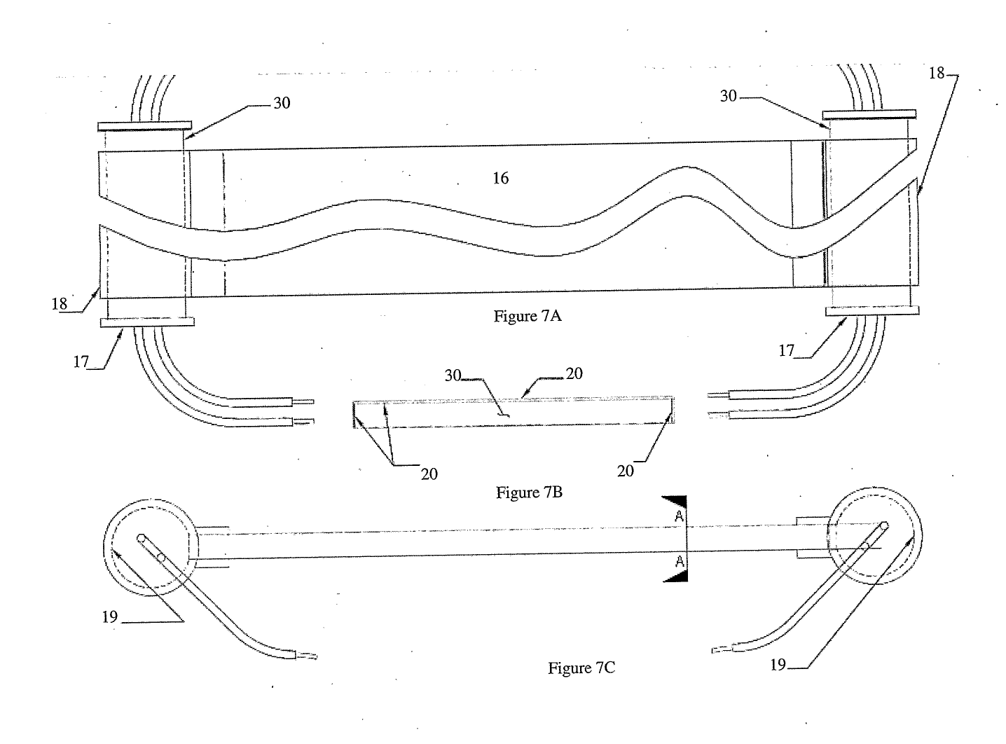

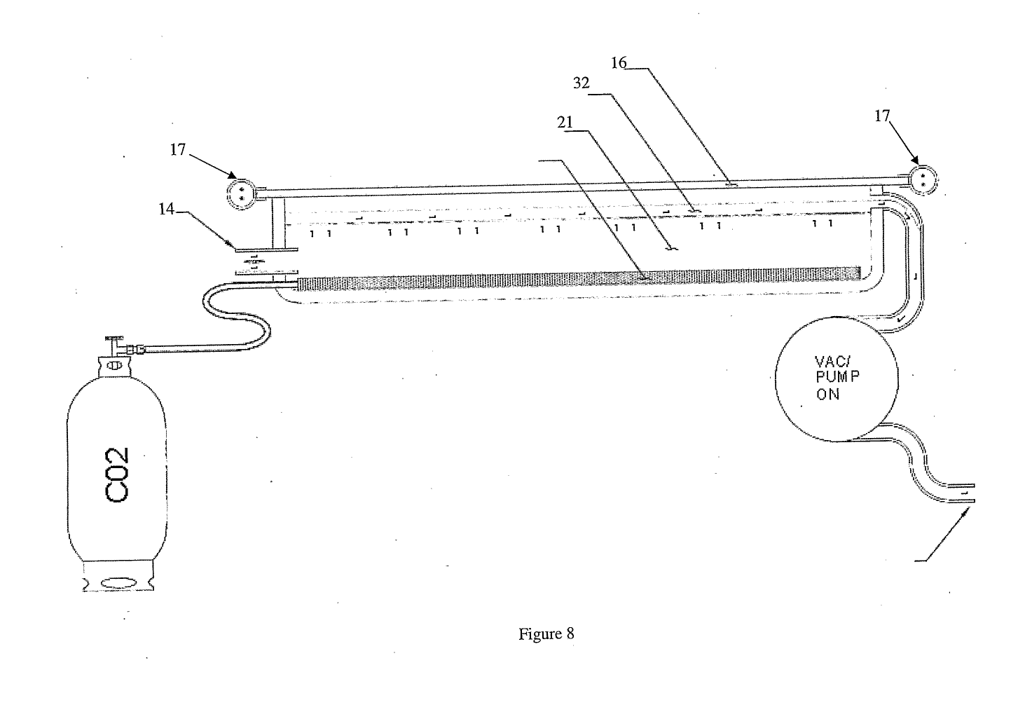

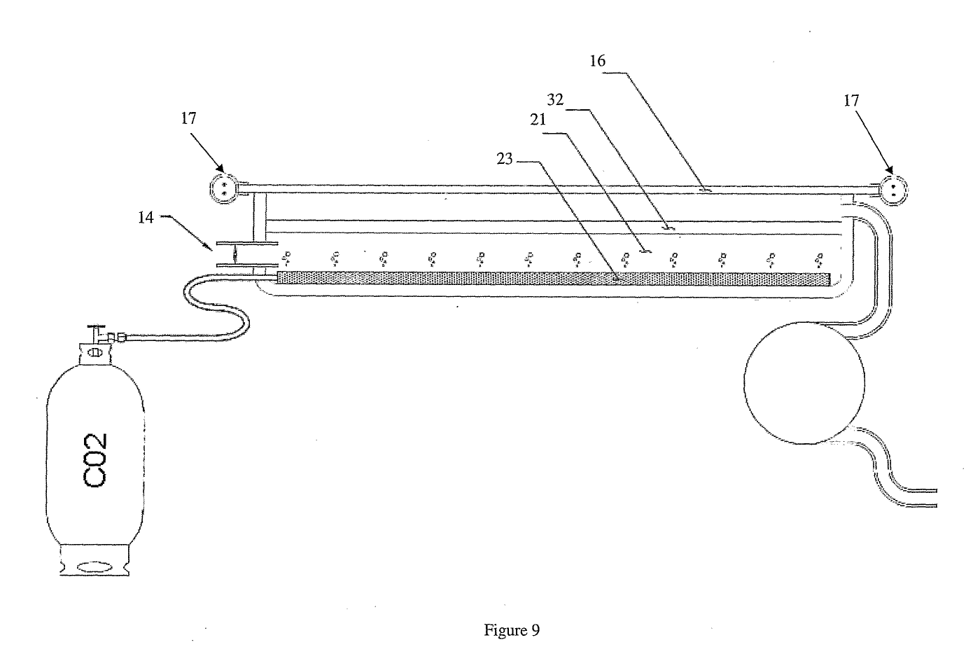

As mentioned above, the outlet 15 is typically substantially opposite the inlet 14. In the preferred rectangular containing vessel configuration illustrated best in FIG. 4, the outlet 15 is located approximately midway along one of the sidewalls 13 of the containing vessel 11 and the inlet 14 is located in a corner of the opposite wall 13 and directed substantially parallel with that wall 13 in order to create a vortex effect when the inlet 14 is open and liquid is entering the containing vessel 11 through the inlet 14.

The outlet 15 is located through an upper portion of a sidewall. The outlet 15 is located on the suction side of a pump 27, connected using a conduit 28 in order to assist outflow of material from the containing vessel. The expulsion side of the pump is also provided with a conduit 29 to transfer the extracted biomass to filtration and holding tanks (not shown).

The bioreactor of the illustrated embodiment is provided with an upper wall or lid 16 to close the bioreactor in order to optimise growth of the biological material. However, the interior of the containing vessel 11 should be accessible for maintenance for example and therefore, the upper wall or lid 16 is removable from the containing vessel.

The upper wall or lid 16 is shaped to match the shape of the containing vessel 11 and overlies the sidewalls 13 of the containing vessel.

The upper wall or lid 16 is manufactured of a transparent or translucent material such as a plastic material. Acrylic plastic is particularly preferred due to its high transmissivity of electromagnetic radiation and particularly light.

Light scattering agents 30 are incorporated into the upper wall or lid 16, normally embedded in the material used to manufacture the upper wall or lid 16.

According to the illustrated embodiment, the upper surface and the edges of the upper wall or lid 16 are provided with a reflective material in order to contain and direct light from the light source 17 into the containing vessel 11.

In the illustrated embodiment, a light tube 18 is provided at one side of the rectangular upper wall or lid 16 and the other three edges of the upper wall or lid 16 are provided with the reflective material. The light tube 18 is provided at a level which is coplanar with the upper wall or lid 16.

Although not illustrated, according to a particularly preferred form, the light source 17 includes a plurality of coloured light emitting diodes mounted on a circular support, the light emitting diodes chosen to provide predetermined spectral wavelengths and an optical device to transmit the emitted light. The support of the light source 17 is an end cap for the light tube 18.

The lighting source of the present invention is normally a powered electrical apparatus and as such, requires electrical connections to a power source. The electrical connections 31 normally extend into the support from a suitable power supply.

The light tube 18 extends along one edge of and abutting the upper wall or lid 16 where the upper wall or lid is planar, as illustrated.

The light tube 18 is provided with reflective portions to allow the transmission of light along the length of the tube and at least one section of the light tube 18 lacks reflective material in order to allow the light to be directed outwardly through the at least one section which abuts one edge of the upper wall or lid 16.

The light tube 18 illustrated is cylindrical and a portion of the light tube without reflective material abuts an edge of the upper wall or lid 16 to transmit light from the light tube 18 into the thickness of the upper wall or lid 16.

As illustrated, the light source 17 is located at one end of an elongate cylindrical, hollow light tube 18 and the opposite end of the light tube will normally be capped with reflective material.

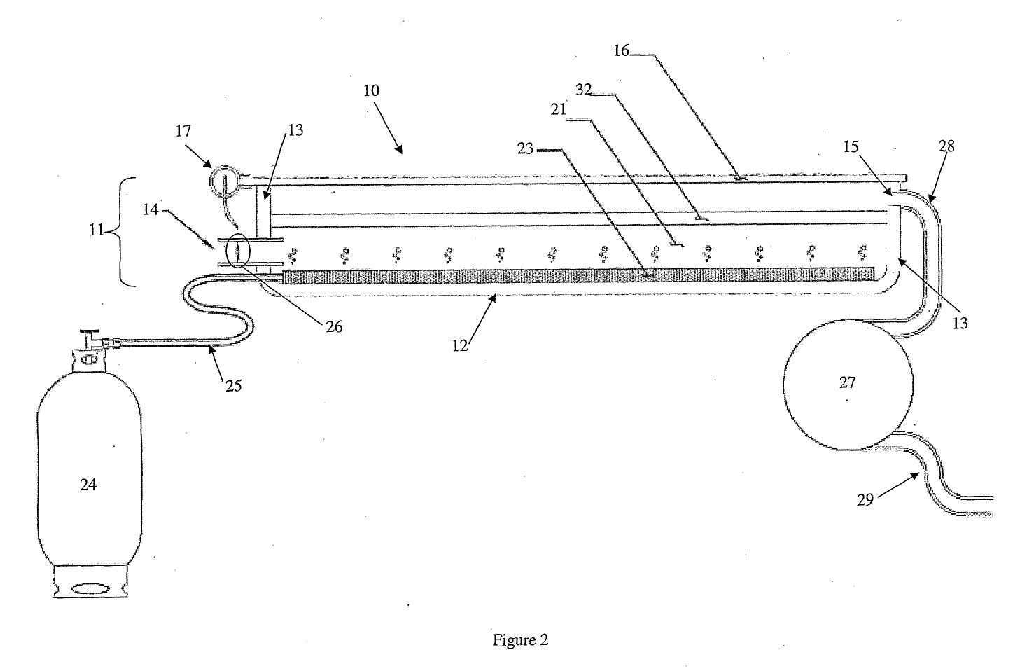

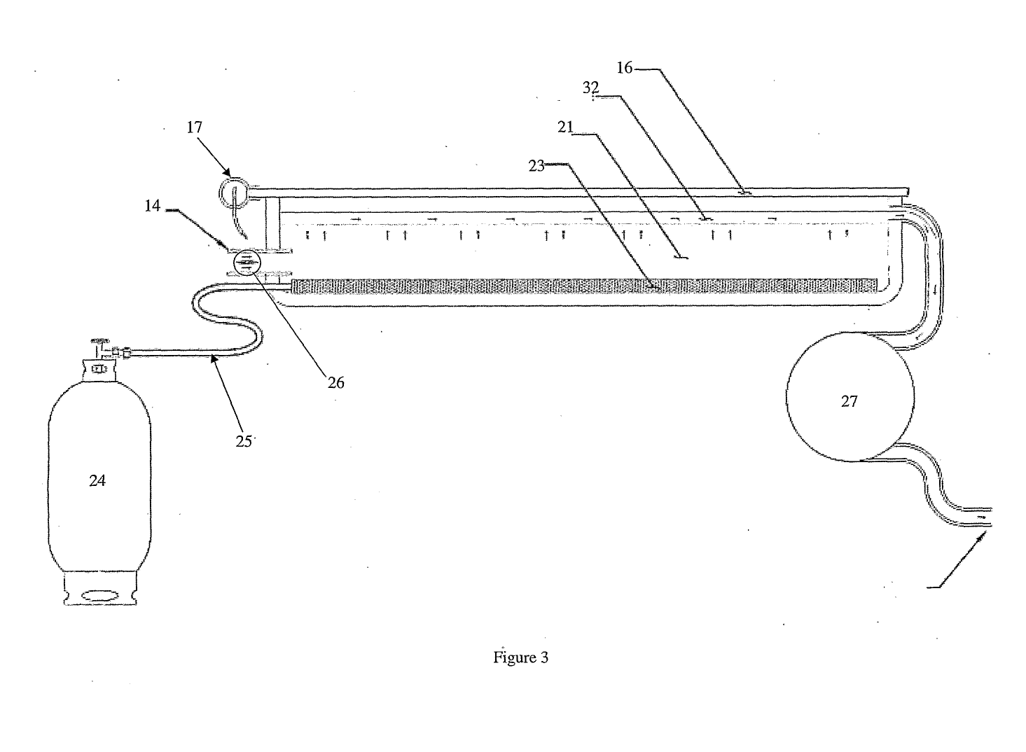

The bioreactor of the present invention is preferably operable in two modes, namely a growth mode illustrated in FIG. 2 and an extraction mode illustrated in FIG. 3.

In the growth mode, the valve 26 in the inlet 14 to the containing vessel 11 is closed. Gas or other material beneficial to the growth of the biological material is bubbled through the liquid growth media 21 contained in the containing vessel 11 via the porous mat 23. The level of liquid growth media 21 and any biomass layer 32 in the containing vessel 11 is below the level of the outlet 15. The outlet pump 27 is off. In this configuration, the biological material which grows in the containing vessel 11, stimulated by the provision of light from the light source, 17 will typically float on the liquid growth media 21. Once a satisfactory amount of the biological material has accumulated in the floating biomass layer 32, the bioreactor 10 is switched to the extraction mode.

In the extraction mode, the gas supply to the porous mat 23 is stopped and the light source 17 may also be switched off The inlet 14 is opened and liquid, normally water will be pumped into the containing vessel 11 to raise the level in the vessel 11. Normally, this will be done at a low flow rate in order to minimise disruption to the biomass layer 32. As the biomass layer 32 reaches the level of the outlet 15, the outlet pump 27 is activated in order to draw the biomass layer 32 from the top of the liquid growth media 21. Minimal liquid is removed in the extraction mode.

Once extraction of the biomass layer 32 has been completed, a portion of the liquid in the containing vessel 11 is drained in order to lower the level below the outlet 15 in order to facilitate growth of the biomass layer once more. The bioreactor 10 then typically enters the growth mode again. By cycling through the growth mode and extraction mode, typically with minimal or no operator interaction, the bioreactor 10 functions through numerous batch growth and extraction modes relatively automatically.

In the present specification and claims (if any), the word “comprising” and its derivatives including “comprises” and “comprise” include each of the stated integers but does not exclude the inclusion of one or more further integers.

Reference throughout this specification to “one embodiment” or “an embodiment” means that a particular feature, structure, or characteristic described in connection with the embodiment is included in at least one embodiment of the present invention. Thus, the appearance of the phrases “in one embodiment” or “in an embodiment” in various places throughout this specification are not necessarily all referring to the same embodiment. Furthermore, the particular features, structures, or characteristics may be combined in any suitable manner in one or more combinations.

1. A bioreactor including a substantially horizontal containing vessel having a base and at least one upstanding sidewall, at least one inlet located at a lower portion of the containing vessel, at least one outlet located at an upper portion of the containing vessel, at least one light source and at least one diffuser, the bioreactor operable in a growth mode in which the at least one inlet to the containing vessel and the at least one outlet are closed, gas or other material beneficial to the growth of the biological material is bubbled through the liquid growth media contained in the containing vessel via the diffuser, and wherein the biological material floats on the liquid growth media as it grows until a satisfactory amount of the biological material has accumulated in the floating layer, and the bioreactor is switched to the extraction mode in which the at last one inlet is opened and liquid is pumped into the containing vessel to raise the level in the vessel such that as the biological material via reaches the level of the at least one outlet, the biological material layer can be removed from the top of the liquid with minimal liquid removed.

2. A bioreactor including a substantially horizontal containing vessel having a base and at least one upstanding sidewall, at least one inlet located at a lower portion of the containing vessel, at least one outlet located at an upper portion of the containing vessel, an upper wall having a reflective material associated there with, at least one light source associated with the upper wall, and at least one light tube associated with each light source extending substantially the length of at least one sidewall of the vessel, the light tube provided adjacent an upper edge of the at least one sidewall and having a reflective lining to direct light from the light source across the upper wall.

3. A bioreactor as claimed in claim 1 wherein the containing vessel is provided with at least one diffuser through which a gas or second liquid enters the containing vessel.

4. a bioreactor as claimed in claim 3 wherein the at least one diffuser is linked to a supply of gas or second liquid which is beneficial to the growth of the biological material.

5. A bioreactor as claimed claim 1 wherein the at least one inlet is located substantially opposite the at least one outlet in order to help flush liquid and/or biomass toward the at least one outlet.

6. A bioreactor as claimed in claim 1 wherein the at least one outlet is located at approximately midway along one of the sidewalls of the containing vessel and the at least one inlet is located in a corner of an opposite wall and directed substantially parallel with that wall in order to create a vortex effect when the at least one inlet is opened and liquid is entering the containing vessel through the at least one inlet.

7. A bioreactor as claimed in claim 1 further including a pump associated with the at least one outlet in order to assist outflow of material from the containing vessel.

8. a bioreactor as claimed in claim 1 further including a secondary outlet located in or through the base in order to drain the containing vessel.

9. A bioreactor as claimed in claim 1 further including an upper wall manufactured of a transparent or translucent material with at least an upper surface and at least some of the edges of the upper wall or lid provided with a reflective material in order to contain and direct light from the at least one light source into the containing vessel.

10. a bioreactor as claimed in claim 9 wherein the at least one light source includes a light tube provided at one side of the upper wall or lid substantially coplanar with the upper wall or lid and any remaining edges of the upper wall or lid are provided with the reflective material as well as the upper surface of the upper wall or lid.

11. A bioreactor as claimed in claim 9 wherein the light tube is provided with reflective portions to allow the transmission of light along the length of the tube and also at least one section of the light tube lacks reflective material in order to allow the light to be directed outwardly through the at least one section.

12. A bioreactor as claimed in claim 1 operable in a growth mode in which the at least one inlet to the containing vessel and the at least one outlet are closed, gas or other material beneficial to the growth of the biological material is bubbled through the liquid growth media contained in the containing vessel via a diffuser, and wherein the biological material floats on the liquid growth media as it grows until a satisfactory amount of the biological material has accumulated in the floating layer, whereupon the bioreactor is switched to an extraction mode.

13. A bioreactor as claimed in claim 12 wherein in the extraction mode the at least one inlet is opened and liquid is pumped into the containing vessel to raise the level in the vessel such that as the biological material via reaches the level of the at least one outlet, the biological material layer can be removed from the top of the liquid with minimal liquid removed.

14. A bioreactor as claimed in claim 13 wherein one extraction of the biological material layer has been completed, a portion of the liquid in the containing vessel is drained in order to lower the level below the at least one outlet in order to facilitate growth of the biological layer once more and the bioreactor is switched to the growth mode again.

15. A bioreactor as claimed in claim 13 wherein cycling through the growth mode and extraction mode occurs with minimal or no operator interaction.

16. (canceled)

17. A bioreactor as claimed in claims 2 wherein the containing vessel is provided with at least one diffuser through which a gas or second liquid enters the containing vessel.

18. A bioreactor as claimed in claim 2 wherein the at least one inlet is located substantially opposite the at least one outlet in order to help flush liquid and/or biomass toward the at least one outlet.

19. A bioreactor as claimed in claim 2 wherein the at least one outlet is located at approximately midway along one of the sidewalls of the containing vessel and the at least one inlet is located in a corner of an opposite wall and directed substantially parallel with that wall in order to create a vortex effect when the at least one inlet is open and liquid is entering the containing vessel through the at least one inlet.

20. A bioreactor as claimed in claim 2 further including a pump associated with the at least one outlet in order to assist outflow of material from the containing vessel.

21. A bioreactor as claimed in claim 2 further including a secondary outlet located in or through the base in order to drain the containing vessel.

22. A bioreactor as claimed in claim 2 further including an upper wall manufactured of a transparent or translucent material with at least an upper surface and at least some of the edges of the upper wall or lid provided with a reflective material in order to contain and direct light from the at least one light source into the containing vessel.

23. A bioreactor as claimed in 2 operable in a growth mode in which the at least one inlet to the containing vessel and the at least one outlet are closed, gas or other material beneficial to the growth of the biological material is bubbled through the liquid growth media contained in the containing vessel via a diffuser, and wherein the biological material floats on the liquid growth media as it grows until a satisfactory amount of the biological material has accumulated in the floating layer, whereupon the bioreactor is switched to an extraction mode.
